SEQUENCE LISTING (2 sequences)

SEQ ID NO: 1 - DNA, 1074 bp, Phanerochaete chrysosporium
  CDS (1)..(1074)
gcagtctgtccagacggcacccgcgtcactaacgcagcttgttgcgcatttattccactggctcaagacctccaggagaccctcttccagggcgactgcggtgaagatgcgcatgaggtcattcgtcttaccttccacgacgccattgctatctctcagagcctgggaccccaggccggcggtggtgctgacggctccatgctgcacttcccgaccatcgagccgaatttttcggcgaacaacggcattgacgactccgtgaacaaccttatccccttcatgcagaagcacaacacgatcagcgccgctgacctcgtccagttcgcgggagccgttgctctcagtaactgccccggcgcccctcgcctcgagttcctcgctggtcgcccgaacacgactattcccgcagtcgagggcctcatccctgagccgcaggacagtgtcaccaaaattctacaacgcttcgaggacgcagggaacttctcgccttttgaggtcgtatccctcctcgcctctcacactgttgctcgtgcagacaaggtcgacgagaccatcgacgccgcacccttcgattccacgcctttcactttcgacacccaggtcttcctcgaggtccttctgaagggtaccggcttccctggatcgaacaacaacaccggtgaggtcatgtccccacttcccctcggcagcggcagcgacacgggcgagatgcgcctgcagtctgactttgcgctcgcgcgcgacgagcgcacggcgtgcttctggcagtcgttcgtcaacgagcaggagttcatggcggcgagcttcaaggccgcgatggcgaagctcgcgatcctcggccacagccgcagcagcctcatcgactgcagcgacgtcgtccccgtgccgaagcccgccgtcaacaagcccgcgacgttccccgcgacgaagggccccaaggacctcgacacactcacgtgcaaggccctcaagttcccgacgctgacctctgaccccggtgctaccgagaccctcatcccccactgctccaacggcggcatgtcctgccctggtgttcagttcgatggccctgcc

SEQ ID NO: 2 - protein, 358 aa, Phanerochaete chrysosporium
AVCPDGTRVTNAACCAFIPLAQDLQETLFQGDCGEDAHEVIRLTFHDAIAISQSLGPQAGGGADGSMLHFPTIEPNFSANNGIDDSVNNLIPFMQKHNTISAADLVQFAGAVALSNCPGAPRLEFLAGRPNTTIPAVEGLIPEPQDSVTKILQRFEDAGNFSPFEVVSLLASHTVARADKVDETIDAAPFDSTPFTFDTQVFLEVLLKGTGFPGSNNNTGEVMSPLPLGSGSDTGEMRLQSDFALARDERTACFWQSFVNEQEFMAASFKAAMAKLAILGHSRSSLIDCSDVVPVPKPAVNKPATFPATKGPKDLDTLTCKALKFPTLTSDPGATETLIPHCSNGGMSCPGVQFDGPA

(19) United States
(12) Patent Application Publication
(10) Pub. No.: US 2003/0113835 A1
(43) Pub. Date: Jun. 19, 2003

(54) Methods for protein synthesis, protein screening and retrieval of protein function

(75) Inventors: Chie Imamura, Aichi (JP); Haruo Takahashi, Aichi (JP); Hideo Nakano, Aichi (JP); Tsuneo Yamane, Aichi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOYOTA CHUO KENKYUSHO, Aichi-gun (JP)

(21) Appl. No.: 10/211,571

(22) Filed: Aug. 5, 2002

(30) Foreign Application Priority Data
Aug. 6, 2001 (JP) 2001-237467

(51) Int. Cl. C12P 21/06

(52) U.S. Cl. 435/68.1; 435/69.1

(57) ABSTRACT

A method for protein synthesis for efficiently preparing a protein with intermolecular and/or intramolecular disulfide bonds synthetically, using a cell-free extract solution to which a template gene is added. The protein with intermolecular and/or intramolecular disulfide bonds is preferably an antibody or a metalloprotein. The method more preferably includes adding a protein with the chaperone function to the cell-free extract solution and/or eliminating reducing agents from the cell-free extract solution. A method for protein screening and a retrieval method for protein function, using this method for protein synthesis. The method provides a method for protein synthesis capable of efficiently preparing a protein with intermolecular and/or intramolecular disulfide bonds synthetically by cell-free systems (in vitro transcription/translations systems).

### BACKGROUND OF THE INVENTION

1. Field of the Invention

The present invention relates to a method for protein synthesis, a method for protein screening and a method for retrieval of protein function.

More specifically, the present invention relates to a method for efficient synthesis of a protein with specific intermolecular and/or intramolecular disulfide bonds, using cell-free protein synthesis systems (in vitro transcription/translation systems). Further, the invention relates to a method for protein screening using this method for protein synthesis. Still further, the invention relates to a method for retrieval of protein function using this method for protein synthesis.

2. Description of the Related Art

In recent years, large-scale DNA analyses have been made, including the elucidation of the nucleotide sequences of the Oryza genome and the human genome. A big factor enabling these analyses was the development of PCR (polymerase chain reaction), enabling rapid cloning of template DNA in large amounts.

Currently, research is in the so-called “post-genome era”. Great attention has been focused on the analysis of functional proteins such as various enzymes and antibodies as expressed via the transcription and translation of genes in genomes. A functional protein should desirably be produced on such a large scale that the protein can be analyzed. However, there has been no large-scale synthesis technique of protein via self replication equivalent to PCR for DNA yet.

Therefore, an efficient method for protein synthesis via cell-free protein synthesis systems (in vitro transcription/translation systems) is now drawing attention. In this method, by using a cell-free extract solution, the elements essentially required for the process of transcribing and translating a template gene can be maintained. In this way, diverse restrictions involved in intracellular protein synthesis can be removed. For certain reasons, cell-free extract solutions derived from for example Escherichia coli and wheat germ cell are favorably used.

However, the content of the process of intracellular protein synthesis and differences in this process among various biological species have not been elucidated in detail. Furthermore, there are still many points to be elucidated as to whether there are differences in the process between protein synthesis in a cell-free system and that in the original cell, and if any, what are the differences. Assume for example that the success in the efficient synthesis of a specific functional protein of active form is reported. In this case, it can be assumed that a similar functional protein of the same family as the above functional protein can also be synthesized in the same cell-free system. However, it is difficult to predict the synthesis of a protein of a type different from the above specific functional protein.

Proteins with intermolecular and/or intramolecular disulfide bonds include very useful proteins such as various antibodies and specific functional proteins containing metals (for example, enzymes containing metals). Antibodies are functional proteins with unique spatial conformations made with intermolecular disulfide bonds formed between L chains and H chains. The family of functional proteins having intermolecular and/or intramolecular disulfide bonds and containing metals includes: peroxidase, an enzyme containing iron (Fe); hemoglobin, an oxygen transport protein containing Fe as well; hemocyanin, an oxygen transport protein containing copper (Cu); and calmodulin, a signal transduction-related protein bound with calcium.

However, it is generally said that it is difficult to synthetically prepare active forms of these proteins in cell-free systems.

For example, there have been no reports of the synthesis of proteins with intermolecular disulfide bonds, such as antibodies, in cell-free systems. There is an example of the synthesis of an artificial active-form antibody, produced by changing a Fab antibody whose H chain and L chain have been crosslinked with intermolecular disulfide bonds into a single chain by linking together these H and L chains with peptide linkers (Nat. Biotechnol. vol.15, p.79, 1997). However, this is not a protein with intermolecular disulfide bonds.

An example of the synthesis of the recombinant type heme protein peroxidase as an inclusion body in Escherichia coli has been reported. In this case, it is necessary to solubilize the inclusion body with a denaturing agent such as urea and then add heme for refolding (Tien et al., Biochem. Biophys. Research Comm.; 216, 1013-1017; 1995). Amold et al. report an example of the secretion of an active form of horseradish peroxidase (HRP) enzyme in the periplasm of Escherichia coli (Biotechnol. Prog.; 15, 467-471; 1999) and an example of the expression and secretion of HRP in yeast (Protein Eng.; 13, 377-84; 2000). In these cases, it is required to add a heme precursor (δ-aminolevulinic acid) to the culture medium. An example of the expression and secretion of CiP (Coprinus cinereus peroxidase) in yeast is also reported (Novo Nordisk; Nature biotechnol.; 17, 379-384; 1999).

However, cell-free systems are not used in any of the above examples of metalloprotein synthesis. In case of the synthesis of metalloproteins such as heme-containing protein as an inclusion body in Escherichia coli, generally, proteins of inactive form are obtained. In order to refold the inactive form into active form, a time period of several days is needed for the processing, which is a hindrance to production. Expression systems using yeast cells are not efficient in operation due to the slow growth of yeast. Even in case of metalloprotein synthesis in cells of higher animals and plants intrincically capable of generating metalloproteins, synthesis of a large amount of metalloprotein is generally thought to be difficult because of the toxicity of metal-containing substances which must be added in large amounts.

Thus, a method for rapid and large-amount synthesis of active form proteins with intermolecular and/or intramolecular disulfide bonds, specifically (a) active form antibodies and (b) active form metalloproteins is desired. Further, such method is also desired to obtain novel modified enzymes by screening in directed evolution as an important technique for the post-genome era. Still further, such method is strongly desired in view of applications to so-called bio-informatics.

It has been expected that there is provided an efficient method for such protein synthesis using cell-free protein synthesis systems (in vitro transcription/translation systems). However, no report giving any example of successful methods for synthesis of the proteins (a) and (b) above has been published yet.

Up to now it has not been known whether the proteins (a) and (b) can be synthetically prepared in cell-free systems. It has not been known, either, whether proteins (a) and (b) of active form can be synthetically prepared in cell-free systems. It has not been known whether the proteins (a) and (b) of active form can be synthetically prepared in versatile cell-free systems derived from Escherichia coli and the like. Further, it has not been known whether the proteins (a) and (b) of active form can be synthetically prepared in large amounts efficiently in cell-free systems.

### SUMMARY OF THE INVENTION

It is an object of the invention to provide a method for protein synthesis, which can efficiently produce a protein with intermolecular and/or intramolecular disulfide bonds in a cell-free system. These proteins are preferably antibodies or metalloproteins. It is another object of the invention to provide a method for protein synthesis, which can efficiently produce the protein of active form in a cell-free system. It is a further object of the invention to provide a method of screening these proteins using this method for protein synthesis. It is a still further object of the invention to provide a method for retrieval of protein function using this method for protein synthesis.

In a first aspect, the invention provides a method for protein synthesis, comprising adding a template gene of a protein with intermolecular and/or intramolecular disulfide bonds to a cell-free extract solution, and synthetically preparing the protein with intermolecular and/or intramolecular disulfide bonds using the cell-free extract solution.

As in the first aspect, the present inventors have achieved the first success in the synthesis of a protein with intermolecular and/or intramolecular disulfide bonds via a cell-free protein synthesis system (in vitro transcription/translation system) by adding a given template gene to a cell-free extract solution.

Because such cell-free extract solution is used in the first aspect, diverse restrictions involved in intracellular protein synthesis can be removed, while essential elements required for the process of the intracellular transcription and translation of a template gene are maintained. Thus, the desired protein can be produced rapidly in large amounts.

In a second aspect of the present invention, at least one protein with the chaperone function is added together with the template gene to the cell-free extract solution in the first aspect.

The second aspect provides one of the most notable aspects of the invention, i.e. the addition of a protein having the chaperone function, together with a template gene, to the cell-free extract solution. This remarkably improves the yield of an active form protein with intermolecular and/or intramolecular disulfide bonds.

Conventionally, there has been no successful examples of synthesis of an active form protein with intermolecular and/or intramolecular disulfide bonds via cell-free systems, and the second aspect has demonstrated for the first time that it is possible. The addition of a protein with the chaperone function together with a template gene to cell-free extract solutions enables exact synthesis and folding of a peptide chain on the basis of the template gene, and promotes efficiently its conversion into the active form protein.

In a third aspect of the invention, the protein with the chaperone function in the second aspect is protein disulfide isomerase (PDI).

As the protein with the chaperone function to be used in accordance with the invention, PDI specified in the third aspect is particularly preferable.

In a fourth aspect of the invention, the protein with the chaperone function in the second aspect is DnaK, DnaJ, GroEL, GroES, GrepE or thioredoxin (Tx).

As the protein with the chaperone function to be used in accordance with the invention, the various proteins in the fourth aspect can be favorably used.

In a fifth aspect of the invention, reducing agents are eliminated from the cell-free extract solutions in the first to fourth aspects.

One of the points of particular interest in accordance with the invention resides in that attention has been paid to reducing agents such as DTT (dithiothreitol) which are routinely added to cell-free extract solutions. In order to express the function of a protein with intermolecular and/or intramolecular disulfide bonds, a specific pattern of intermolecular and/or intramolecular disulfide bonds must be formed. However, the present inventors have found that such specific pattern of disulfide bonds are difficult to form reliably when cell-free extract solutions are in reduced state. Thus, the elimination of reducing agents from cell-free extract solutions is very effective for the synthesis of a variety of active form proteins requiring the formation of intermolecular and/or intramolecular disulfide bonds to express the function thereof.

Herein, the yield of an active form protein with intermolecular and/or intramolecular disulfide bonds can be far more greatly improved by adding protein with the chaperone function to cell-free extract solutions and eliminating reducing agents from the cell-free extract solutions.

In a sixth aspect of the invention, the protein with intermolecular and/or intramolecular disulfide bonds in accordance with the first to fifth aspects is an antibody.

Among “proteins with intermolecular and/or intramolecular disulfide bonds”, an antibody is a particularly good example of a protein with intermolecular disulfide bonds. The inventors have succeeded for the first time in the production of an antibody via a cell-free protein synthesis system (in vitro transcription/translation system), by adding a given template gene to a cell-free extract solution.

In a seventh aspect of the invention, the antibody of the sixth aspect is obtained in a cell-free extract solution, in an active form, at a concentration such that the activity of the antibody can be assayed as it is in the cell-free extract solution. Concerning the active form antibody, the term “concentration such that the activity of the antibody can be assayed as it is in the cell-free extract solution” means that the amount of the antibody of active form as synthesized in the cell-free extract solution is above 10 μg/mL.

It has been found that by the method for the synthesis of the protein in the sixth aspect, the antibody of active form can be obtained in a cell-free extract solution at a concentration such that the activity of the antibody can be assayed as it is in the cell-free extract solution, as in the seventh aspect. Thus, the activity of the antibody as a desired protein can be assayed without concentration or purification procedures of the antibody. This effect brings about great merits in that rapid large-scale screening can be carried out, using automatic devices and the like at sites of research and development, because protein functions such as the activity can generally be assessed when a transcription/translation reaction solution is at 1 μl.

In an eighth aspect of the invention, the protein with intermolecular and/or intramolecular disulfide bonds in the first to fifth aspects is a metalloprotein.

Among “proteins with intermolecular and/or intramolecular disulfide bonds”, a metalloprotein is a particularly preferable example of a protein with intramolecular disulfide bonds. The inventors have succeeded for the first time in the production of a metalloprotein via a cell-free protein synthesis system (in vitro transcription/translation system) using a template gene.

The method for metalloprotein synthesis can generally be applied to the metalloprotein family including: an iron (Fe)-containing enzyme peroxidase; an oxygen transport protein hemoglobin containing Fe; an oxygen transport protein hemocyanin containing copper (Cu); a calcium-bound signal transduction-related protein calmodulin; and the like.

In a ninth aspect of the invention, a functional unit containing a metal is added together with the template gene to the cell-free extract solution in the eighth aspect.

In the eighth aspect, it is preferable to add a functional unit containing a metal together with the template gene to the cell-free extract solution.

In a tenth aspect of the invention, at least a part of the metalloprotein in the ninth aspect is obtained in the active form with a specific function in which the functional unit containing a metal is involved.

It has been confirmed that according to the method for protein synthesis in the ninth aspect, at least a part of a metalloprotein is obtained in the active form with a specific function in which the functional unit containing a metal is involved. Therefore, it is possible to avoid time consuming for folding and other operations to activate the metalloprotein after it has been obtained.

In an eleventh aspect of the invention, the functional unit containing a metal in the ninth aspect is a metal complex.

In the ninth aspect, the functional unit containing a metal to be added together with a template gene to a cell-free extract solution, is preferably a metal complex.

In a twelfth aspect of the invention, the metal complex in the eleventh aspect is hemin.

As the metal complex in the eleventh aspect, particularly, hemin is a preferable example. Conventionally, there is an example that a metal (Fe) ion and a heme precursor (δ-aminolevulinic acid) for example were added to a culture medium so as to generate a metalloprotein as an inclusion body, as in the related art described above. However, it has been found for the first time that the addition of a functional unit containing a metal, such as a metal complex, together with a template gene enables to produce a metalloprotein in a cell-free extract solution.

In a thirteenth aspect of the invention, the metalloprotein in the eighth to twelfth aspects is a heme-containing enzyme.

As the metalloprotein synthetically prepared in accordance with the eighth to twelfth aspects, heme-containing enzymes such as various peroxidases are particularly useful.

Uses for the above include detergents, monomer polymerization, pulp bleaching in non-chlorine systems, decomposition of persistent environmental pollutants such as aromatic compounds, and liquid waste disposal.

In a fourteenth aspect of the invention, the heme-containing enzyme in the thirteenth aspect is peroxidase.

As the heme-containing enzyme in the thirteenth aspect, various peroxidases are preferably exemplified.

In a fifteenth aspect of the invention, the metalloprotein in accordance with the eighth to fourteenth aspects is obtained in the active form in a cell-free extract solution at a concentration such that its activity can be assayed as it is in the cell-free extract solution. The term “concentration such that its activity can be assayed as it is in the cell-free extract solution” concerning the metalloprotein of active form means that the amount of the active form metalloprotein synthetically prepared in the cell-free extract solution is above 10 μg/mL.

It has been found that by the method for protein synthesis in accordance with the eighth to fourteenth aspects, an active form metalloprotein as in the fifteenth aspect is obtained in a cell-free extract solution at a concentration such that its activity can be assayed as it is in the cell-free extract solution.

Thus, the activity of the metalloprotein as a desired protein can be assayed without concentration or purification procedures of the protein. The effect brings about great merits in that rapid large-scale screening can be carried out using automatic devices and the like at sites of research and development, because protein functions such as the activity can generally be assessed when the transcription/translation reaction solution is at 1 μl.

In a sixteenth aspect of the invention, the cell-free extract solution in accordance with the first to fifteenth aspects is derived from Escherichia coli.

The cell-free extract solution derived from Escherichia coli is widely used and readily available. It is very advantageous for the practice of the invention to synthetically prepare proteins with intermolecular and/or intramolecular disulfide bonds, such as antibodies and metalloproteins, using cell-free extract solutions derived from Escherichia coli.

In a seventeenth aspect of the invention, a method for protein screening is provided, comprising: adding to a cell-free extract solution a template gene prepared by subjecting the nucleotide sequence of the nucleic acid encoding an arbitrary type of protein with intermolecular and/or intramolecular disulfide bonds to a specific or random modification; synthetically preparing a protein encoded by the template gene using the cell-free extract solution; and screening the synthetically prepared protein by assaying the activity.

Attention is now being paid to an approach for screening a protein with a new function, including subjecting the nucleotide sequence of the nucleic acid encoding a protein of an arbitrary type to a specific or random modification and expressing the resulting nucleotide sequence as the template gene.

In the seventeenth aspect, the approach can be applied to a protein with intermolecular and/or intramolecular disulfide bonds in cell-free systems. The method is far more effective in cases where a protein with the chaperone function is added to a cell-free extract solution and/or in cases that reducing agents are eliminated from the cell-free extract solution. Concerning such protein, consequently, an active form protein can be synthetically prepared in a rapid fashion to enable the large-scale screening of a protein with a new function. For example, such screening which requires nearly one month in systems using viable cells such as Escherichia coli can be carried out only in one day.

In an eighteenth aspect of the invention, a retrieval method for protein function is provided, comprising adding to a cell-free extract solution a template gene encoding an arbitrary protein requiring the formation of a specific pattern of intermolecular and/or intramolecular disulfide bonds for the expression of a function, where at least a part of the function is unknown; synthetically preparing the protein encoded by the template gene using the cell-free extract solution; and retrieving the unknown function of the protein by testing the function of the prepared protein.

The retrieval of the functions of an appropriate protein group requiring the formation of a specific pattern of intermolecular and/or intramolecular disulfide bonds for the expression of the functions, where at least a part of the functions is unknown, is very important in the post-genome era. Therefore, these proteins should be expressed in their active forms in a rapid manner and on at least a production scale enabling its analysis.

However, the protein synthesis in conventional in vitro transcription/translation systems involved difficulty in the synthesis of active form protein. In accordance with the eighteenth aspect, the problem described above can be overcome to provide an efficient retrieval method for protein function. The method is far more effective in case that a protein with the chaperone function is added to a cell-free extract solution and/or in case that reducing agents are eliminated from the cell-free extract solution.

In a nineteenth aspect of the invention, the protein in the eighteenth aspect is a protein composing a subunit of a protein complex with an appropriate function.

As the protein in accordance with the eighteenth aspect, a protein composing the subunit of a protein complex with an appropriate function is preferable. The protein composing the subunit of a protein complex is joined with a protein molecule composing another subunit by a specific pattern of intermolecular disulfide bonds.

In a twentieth aspect of the invention, the protein complex in accordance with the nineteenth aspect is an antibody.

As the protein complex in accordance with the nineteenth aspect, antibodies are preferable examples. In such antibodies, the specific pattern of intermolecular disulfide bonds is formed between the L chain and the H chain.

The above and other advantages of the invention will become more apparent from the following description and the accompanying drawings.

### BRIEF DESCRIPTION OF THE DRAWINGS

FIG. 1 shows graphs depicting the assay results of the MnP activity in Example 1-1.

FIG. 2 shows graphs depicting the assay results of the MnP activity in Example 1-2.

FIG. 3 shows graphs depicting the assay results of the MnP activity in Example 1-2, without any purification or concentration of MnP.

FIG. 4 is a photo depicting the results of Western blotting in Example 3.

FIG. 5 shows three-dimensional graphs depicting the results of the detection of positive clones with HTS in Example 4.

FIG. 6 shows graphs depicting the assay results of the MnP activity of the primary positive clones in Example 4.

FIG. 7 shows graphs depicting the results of the screening of mutated MnP in Example 5.

FIG. 8 shows graphs depicting the assay results of the activity of mutated MnP in Example 5.

FIG. 9 shows graphs depicting the assay results of the LiP activity in Example 2.

### DETAILED DESCRIPTION OF THE INVENTION

(Method for Protein Synthesis)

The method for protein synthesis in accordance with the invention is a method for protein synthesis via in vitro transcription/translation systems for the synthesis of a protein with intermolecular and/or intramolecular disulfide bonds. The invention is practiced by adding at least a template gene to a cell-free extract solution. In case of the synthesis of the metalloprotein to be described below, a functional unit containing the metal found in the metalloprotein is preferably added together with the template gene to the cell-free extract solution.

The method for protein synthesis in accordance with the invention is particularly preferably practiced in the following embodiments (c) to (e).

(c) Addition of at least one protein with the chaperone function together with the template gene to the cell-free extract solution.

(d) Elimination from the cell-free extract solution reducing agents such as DTT routinely used therein. The term “elimination of reducing agents” means that reducing agents such as DTT routinely used for in vitro transcription/translation systems are never added, or that reducing agents already added to commercially available in vitro transcription/translation systems are eliminated via dialysis, or that SH residue modifiers such as iodoacetamide or oxidants are added to consequently render reducing agents ineffective.

(e) The addition of proteins with the chaperone function as in (c) is simultaneously done with the elimination of reducing agents as in (d).

In these embodiments (c) to (e), the active form protein in the cell-free extract solution can be obtained in the cell-free extract solution at a concentration such that the activity can be assayed with no need of concentration or purification procedures, while the protein is still in the cell-free extract solution.

(Cell-free Extract Solution)

As the cell-free extract solution, widely used cell-free extract solutions derived from common Escherichia coli, wheat germ, and insect cells can of course be used. However, the cell-free extract solution is not limited to them. Cell-free extract solutions derived from general prokaryotic organisms such as bacteria, general eukaryotic unicellular organisms such as yeast, general plant cells, and general animal cells can be used. For the method for metalloprotein synthesis, cell-free extract solutions derived from cells originally having the potency to generate such metalloprotein as well as cell-free extract solutions derived from cells originally not having the potency to generate such metalloprotein can be used. Cell-free extract solutions derived from cells without any pathway for the synthesis of a metal complex from the metal ion and a precursor can also be used.

As the method for preparing a cell-free extract solution from a variety of the cells, various well known or known methods may appropriately be selected, depending on the need, with no specific limitation. For example, cells can be ground with homogenizers; cells can be disrupted by a rapid pressure change or by an ultrasonic process. In addition to these procedures, cytoskeleton and the like may satisfactorily be removed by centrifugation or the like. In addition, commercially available cell-free extract solutions can be used. In case of commercially available products with reducing agents such as DTT preliminarily added thereto, the reducing agents are preferably eliminated or rendered ineffective as described above.

Various components may preferably be added to the cell-free extract solution so as to improve the potency of protein synthesis. Such components include buffers, four types of nucleotide triphosphates and RNA polymerase for use in the transcription of template gene, various amino acids for use in translation, transfer RNA (tRNA), polyethylene glycol, and phosphorylation enzymes.

(Template Gene)

As long as the template gene contains a nucleic acid encoding the desired protein with intermolecular and/or intramolecular disulfide bonds, the composition thereof is not limited. Preferably, an appropriate regulatory sequence is linked to the nucleotide sequence of the nucleic acid. For example, a promoter is added to the upstream, while a terminator is added to the downstream. An appropriate vector inserted with the gene starting from the promoter to the terminator can also be used, or fragments of the gene from the promoter to the terminator amplified by PCR can also be used.

(Protein with Specific Intermolecular and/or Intramolecular Disulfide Bonds)

In accordance with the invention, the term “protein with specific intermolecular and/or intramolecular disulfide bonds” means a protein with a specific spatial conformation that is produced by a specific disulfide bond formed intermolecularly or intramolecularly, this bond thus being indispensable for the expression of the function of the protein.

The type of such species of protein is not limited. As the protein with intermolecular disulfide bonds, antibodies with intermolecular disulfide bonds formed between the L chain and the H chain, insulin and α-chymotrypsin are representative. Notable proteins with intramolecular disulfide bonds include various metalloproteins described below, reducing enzymes such as NOx reductase, trypsin, and ribonuclease A.

(Antibody)

In accordance with the invention, the term antibody means a protein with intermolecular and/or intramolecular disulfide bonds and with a given functional unit including the H chain and the L chain as the constitutional element. Types of antibody include proteins with one or two antigen-binding sites such as IgG immunoglobulin antibody, Fab antibody, Fab′ antibody, and F(ab′)₂ antibody.

(Metalloprotein)

In accordance with the invention, the term metalloprotein means a protein with intermolecular and/or intramolecular disulfide bonds and with a given functional unit containing a metal as the constitutional element, where the active form thereof has a specific function in which the functional unit is involved. As long as the conditions are satisfied, the type of the metalloprotein, the type of the functional unit containing a metal, and the type of the metal are not limited.

The metalloprotein includes, for example, an enzyme peroxidase and an oxygen transport protein hemoglobin which are heme-containing proteins containing iron (Fe), hemocyanin which is an oxygen transport protein containing copper (Cu), and a signal transduction-related protein bound with calcium (Ca), namely calmodulin.

The peroxidase includes for example manganese peroxidase, horseradish peroxidase, cytochrome c peroxidase, and lignin peroxidase.

The type of the functional unit containing a metal is not limited but is typically a metal complex, including for example hemin, which is a Fe-porphyrin complex, in peroxidase and hemoglobin.

(Protein with Chaperone Function)

The term protein with the chaperone function means a protein with a function restructuring a desired specific protein into the proper steric structure in order to allow the function essentially specific to the desired protein to be expressed. An example of a protein with the chaperone function is one which exerts this function by causing the subject protein to accurately form disulfide bonds (refold disulfide bonds). Other examples include proteins exerting the chaperone function via disaggregation, stabilization of precursor protein, suppression of aggregation and the like, and proteins exerting chaperone-like functions.

In accordance with the invention, the most preferable protein with the chaperone function (or chaperone-like function) is PDI (protein disulfide isomerase), but other types such as DnaK, DnaJ, GroEL and GroES used in the Examples, and GrepE, Tx and the like as well, may be used with no limitation.

(Method for Protein Screening)

In accordance with the method for protein screening of the invention, the method for the synthesis of a protein with intermolecular and/or intramolecular disulfide bonds is applied to the directed evolution molecular engineering of protein.

More specifically, a template gene is prepared by subjecting the nucleotide sequence of the nucleic acid encoding a protein with intermolecular and/or intramolecular disulfide bonds to a specific or random diverse modification. A great number of template gene types thus prepared are transcribed and translated by the method for protein synthesis. Then, using the resulting various proteins thus synthetically prepared which contain active forms in amounts enough to enable the detection of the activities, or in very large amounts, these active form proteins can be screened rapidly on a large-scale.

The method for protein screening in accordance with the invention is particularly preferably carried out in the embodiments (c) to (e). In case that a metalloprotein is the object, further, a functional unit containing a metal in the metalloprotein is preferably added together with a given template gene.

By the method for protein screening in accordance with the invention, screening can be done when several micro liters of a transcription/translation reaction solution is simply prepared, so that screening can be done in a short period of time. Further, a combination for example with a computer model of protein conformation enables efficient designing of the objective protein.

(Retrieval Method for Protein Function)

In the retrieval method for protein function in accordance with the invention, the method for protein synthesis is applied to the retrieval of the function of a protein requiring the formation of a specific pattern of intermolecular disulfide bonds for the expression of the function, this function (or at least a part of the function) being unknown. More specifically, a template gene encoding an arbitrarily chosen protein with unknown function is added to a cell-free extract solution to retrieve the function of the active form protein synthetically prepared. The type of the protein requiring the formation of a specific pattern of intermolecular disulfide bonds for the expression of the function is not limited, but includes for example a protein composing a subunit of a protein complex with an appropriate function. The type of such protein complex is not limited, but includes for example an antibody including L chain and H chain. Examples of proteins to be subjected to the retrieval of function include metalloproteins.

The retrieval method for protein function in accordance with the invention is particularly preferably practiced in the embodiments (c) to (e). In case that a metalloprotein is the subject, further, a functional unit containing a metal in the metalloprotein is preferably added together with a given template gene.

The retrieval method for protein function in accordance with the invention enables easier estimation of the function of a protein whose function has been unknown, which has been a problem in the recent bioinformatics (proteomics), thus giving great industrial advantages.

### EMBODIMENTS

### EXAMPLE 1-1

### Synthesis of Manganese Peroxidase

A synthetic reaction of a heme protein manganese peroxidase with intramolecular disulfide bonds was done. Specifically, a bacterial cell extract solution of S30 derived from Escherichia coli strain A19 was prepared by general preparative methods including homogenizer process and centrifugation. To the bacterial cell extract solution were added a template gene and the following components 1) to 15) inclusive of hemin, for in vitro transcription/translation at 25° C. for 2 hours. No reducing agents such as DTT (dithiothreitol) were added to the bacterial cell extract solution.

1) Template gene: 50 μg/ml PCR reaction product prepared by adding T7 promoter and a ribosome binding site to the upstream of the manganese peroxidase (MnP) gene derived from white rot fungi, and adding T7 terminator to the downstream thereof, the nucleotide sequence of the manganese peroxidase (MnP) gene being given sequence ID No. 1:

2) 10 μM hemin

3) 56.4 mM Tris-acetate buffer, pH 7.4

4) 1.2 mM ATP, 1 mM each of GTP, CTP, UTP

5) 40 mM creatine phosphate

6) 0.7 mM amino acids mixture (a mixture of 20 types of amino acids composing biological proteins)

7) 4.1% (W/W) polyethylene glycol 6000

8) 35 μg/ml folinic acid

9) 0.2 mg/ml Escherichia coli tRNA

10) 36 mM ammonium acetate

11) 0.15 mg/ml creatine kinase

12) 10 mM magnesium acetate

13) 100 mM potassium acetate

14) 10 μg/ml rifampicin

15) 7.7 μg/ml T7 RNA polymerase.

After the transcription/translation, the reaction product was subjected to Western blotting and autoradiography (labeled with ¹⁴C-leucine) by general methods. The presence of a main band with the same molecular weight as MnP was confirmed. The yield of MnP in the bacterial cell extract solution as estimated on the basis of the concentration of the band was about 10 μg/ml.

Further, 200 μl of the bacterial cell extract solution was separated after the transcription/translation continued for 2 hours; using the His-tag added to the synthetically prepared MnP, MnP was adsorbed on His-tag beads for purification and concentration, to assay the MnP activity.

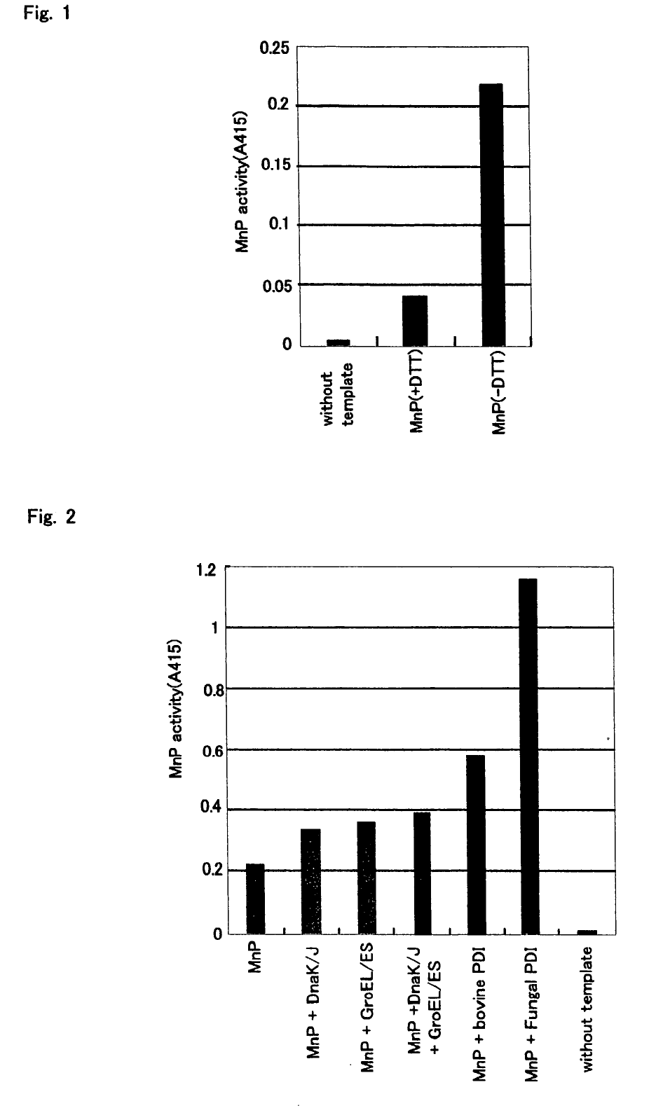

The MnP activity was assayed as follows. A solution (a solution for enzyme activity assay) was prepared by adding 0.1 mM hydrogen peroxide to a solution of 0.5 mM manganese sulfate, 2 mM oxalic acid and 5 mM ABTS (2,2′-azinobis 3-ethylbenzothiazoline-6-sulfonate) in 200 μl of 25 mM succinate buffer, pH 4.5. Then, the beads adsorbing MnP thereon were added to the solution for enzyme activity assay, for reaction at 25° C. for 5 minutes, and then the absorbance at 415 nm was measured. The results are shown in the column “MnP (−DTT)” of FIG. 1. Based on the results, the proportion of active form in the generated MnP was estimated at about 1%.

### Comparative Example

In one comparative example, the protein synthesis in the in vitro transcription/translation system of Example 1-1 was carried out in the same manner as described above except for further addition of a reducing agent DTT of 1.76 mM. In another comparative example, the protein synthesis in the in vitro transcription/translation system of Example 1 was carried out in the same manner as described above except for no addition of the template gene. According to the same procedure, MnP activity of the resultant substances was assayed. The results of the measurement of absorbance are shown in the column “MnP (+DTT)” and the column “without template” in FIG. 1. In any of the comparative examples, the levels of color reactions were extremely low, which indicates the absence of active form MnP.

### Example 1-2

### Effect of Addition of Protein with Chaperone Function

For the protein synthesis in the in vitro transcription/translation system of Example 1-1, chaperonin or PDI shown below in 16) to 20) was respectively added to the cell-free extract solutions. Herein, bovine PDI in 19) and fungus PDI in 20) were added along with 1 mM GSH and 0.1 mM GSSG.

16) 1 μM DnaK and 0.4 μM DnaJ

17) 1.25 μM GroEL and 1.25 μM GroES

18) A mixture of the chaperonins in 16) and 17)

19) 32 μg/ml bovine PDI (commercially available product)

20) 32 μg/ml fungus PDI (as disclosed in Japanese Patent Application Laid-open No.38752/1994)

These examples were subjected to the same procedure as in Example 1-1 above; after reaction, the MnP activity of the MnP adsorbed on the His-tag beads for purification and concentration was measured (absorbance measured) according to the same procedure. The results are shown in FIG. 2. In FIG. 2, the column “MnP” shows the results of Example 1-1; the column “MnP+DnaK/J” shows the results of 16) above; “MnP+GroEL/ES” shows the results of 17) above; “DnaK/J, GroEL/ES” shows the results of 18) above; “MnP+bovine PDI” shows the results of 19) above; “MnP+fungus PDI” shows the results of 20) above; “without template” shows the results of the comparative example with no addition of the template gene as in the Example 1-1.

As is clearly shown in the results of FIG. 2, the effect of the addition of the proteins with the chaperone function on the increase of the ratio of MnP accurately folded into the active form was clearly demonstrated. The effect of the addition of PDI was particularly prominent. The effect of the addition of fungus PDI was prominent in particular. It was estimated that the active form in this case was about 25%.

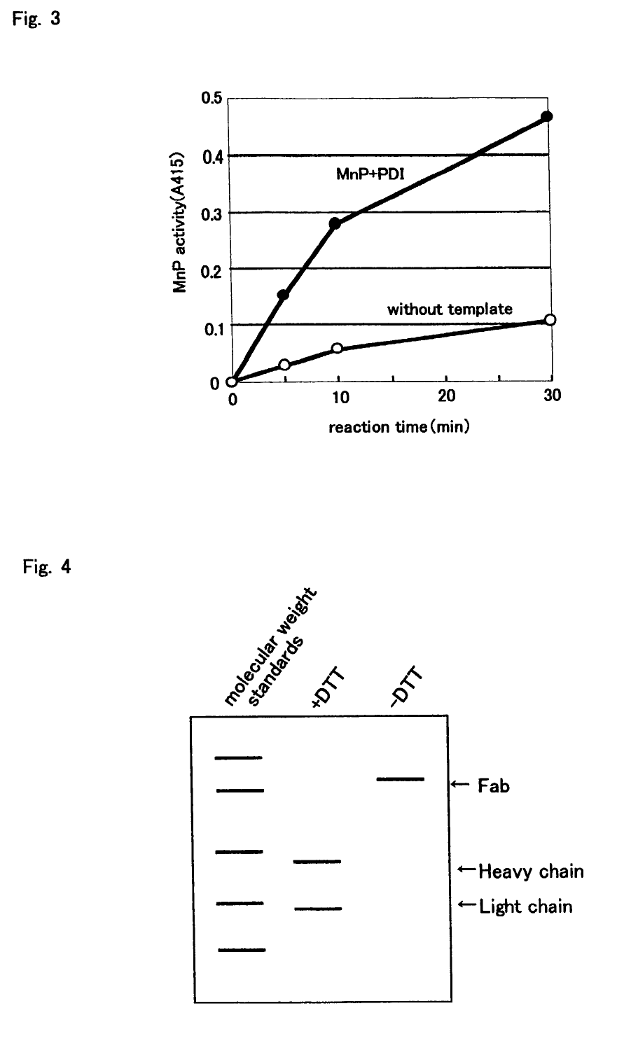

In the example with 20) above among the individual examples, meanwhile, a bacterial cell extract solution resulting from reaction carried out for a given period of time was separated as it was (with no purification or concentration by His-tag beads); then, 1 μl of the bacterial cell extract solution was added to the same solution for enzyme activity assay as in Example 1-1, to assay MnP activity (absorbance measurement). The results are shown in the graph “MnP+PDI” in FIG. 3. The same measurement was done concerning the comparative example without addition of the template gene of the Example 1-1. The results are shown in the graph “without template” in FIG. 3. As is clearly indicated in the graph “MnP+PDI”, the MnP activity in this case can be detected sufficiently.

### EXAMPLE 2

### Generation of Lignin Peroxidase

Under the same conditions as the optimal conditions for MnP Synthesis, protein synthesis of a heme protein lignin peroxidase (LiP) with intramolecular disulfide bonds was done.

Specifically, a LiP template gene of an equal amount as the MnP template gene, the components 2) to 15) as described in Example 1-1, and the component 20) described in Example 1-2 were added to the same bacterial cell extract solution as in Example 1-1. The nucleotide sequence of the LiP template gene was known and is disclosed in GENEBANK/×76689, Nucleic Acids Res (1988) vol.16, page 1219. For the LiP template gene, T7 promoter and a ribosome binding site were added to the upstream, while T7 terminator was added to the downstream, as in the case of the MnP template gene. In vitro transcription/translation reaction was done via the bacterial cell extract solution at 25° C. for 2 hours. No reducing agents including DTT for example were added to the bacterial cell extract solution.

Further, 200 μl of the bacterial cell extract solution just when the in vitro transcription/translation reaction was made for one hour was separated; using the His-tag added to the generated LiP, the LiP was adsorbed on the His-tag beads for purification and concentration, to assay the LiP activity.

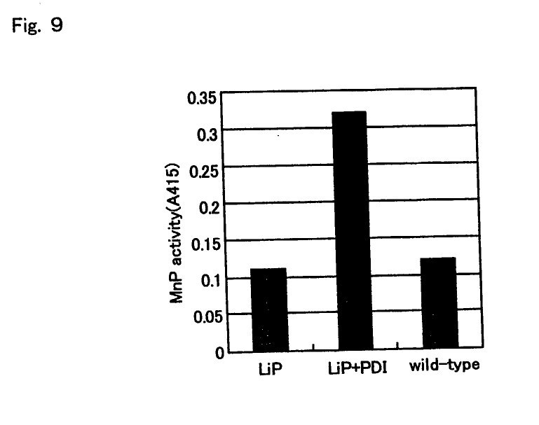

The LiP activity was measured as follows. Specifically, a solution (a solution for enzyme activity assay) was prepared by adding 0.4 mM hydrogen peroxide to a solution of 0.5 mM veratryl alcohol and 5 mM ABTS in 200 μl of 50 mM succinate buffer, pH 4.5. Then, beads adsorbing LiP were added to the solution for enzyme activity assay, for reaction at 25° C. for 5 minutes, and the absorbance at 415 nm was measured. The results are shown in the graph “LiP+PDI” in FIG. 9.

FIG. 9 shows a graph “without template” depicting the results of the same measurement in case of protein synthesis reaction using a bacterial cell extract solution of the same composition, except for no addition of the LiP template gene. FIG. 9 also shows a graph “LiP” depicting the results of the same measurement in case of protein synthesis reaction using a bacterial cell extract solution of the same composition, except for no addition of the component 20). As apparently indicated in FIG. 9, no LiP activity was substantially observed in the graph “LiP” or the graph “without template”. Alternatively, an effective LiP activity was observed in “LiP+PDI”.

### EXAMPLE 3

### Effect of Elimination of Reducing Agents from in vitro Transcription/translation System

Synthesis of the anti-cortisol Fab antibody (with intermolecular S—S bonds) was done under the same conditions as the optimal conditions for MnP synthesis.

Specifically, a template gene of the Fab antibody at an equal amount to the MnP template gene, the components 2) to 15) as described in Example 1-1, and the component 20) described in Example 1-2 were added to the same bacterial cell extract solution as in Example 1-1. The nucleotide sequence of the template gene of the Fab antibody was known and is disclosed in Japanese Patent Application Laid-open No. 139460/2000. In the template gene of the Fab antibody, additionally, T7 promoter and a ribosome binding site were added to the upstream, while T7 terminator was added to the downstream, as in the case of the MnP template gene.

For the in vitro transcription/translation of the Fab antibody, further, a system without any reducing agent added and a system with a reducing agent DTT at 1.76 mM were prepared. In vitro transcription/translation was done in these bacterial cell extract solutions at 25° C. for 2 hours, to prepare the Fab antibody. Subsequently, Western blotting was done as usual. The results are shown in FIG. 4. As is clearly shown in “−DTT” of FIG. 4, generation and activity of the Fab antibody in the system without any reducing agent was detected by comparison through a molecular marker. In the system with DTT added, “+DTT”, active Fab antibody was not generated, due to no formation of intermolecular disulfide bonds between the L chain and the H chain, although these chains were surely generated.

### EXAMPLE 4

### Screening of Mutated MnP Thermally Stabilized

The MnP gene was amplified by error-prone PCR (10 mM Tris-HCl, pH 9.0, 50 mM KCl, 0.1% Triton X-100, 7 mM MgCl₂, 0.7 mM MnCl₂, 0.2 mM dATP, 0.2 mM dGTP, 1 mM dCTP, 1 mM dTTP, 100 ng/μl MnP, 0.3 μM primer, 25 mU/μl Promega Taq DNA polymerase), to prepare a library where 1.5 mutations per 100 bases (error ratio 1.5%) on average were randomly inserted.

After dilution to one molecule/well on average, the following PCR was done, using LA Taq polymerase manufactured by TAKARA SHUZO CO., LTD.:

94° C. for 2 min.;

65 cycles of 96° C. for 10 sec., (Tm-5)° C. for 5 sec., and 72° C. for 1.6 min.; and

72° C. for 7 min.

Subsequently, the components 2) to 15) as described in Example 1-1 and the component 20) described in Example 1-2 were added to the S30 bacterial cell extract solution derived from the Escherichia coli strain A19. Then, 3 μl each of the PCR products were added as template to 37 μl of the bacterial cell extract solution, for in vitro transcription/translation at 25° C. for 3 hours.

Subsequently, 1.5 μl each of the synthesis products were added to 100 μl of the same solution for enzyme activity assay as in Example 1-1, for incubation at 60° C. for 25 minutes, to which was then added hydrogen peroxide to achieve a final concentration of 0.1 mM, to measure MnP activity by the measurement of the absorbance at 415 nm.

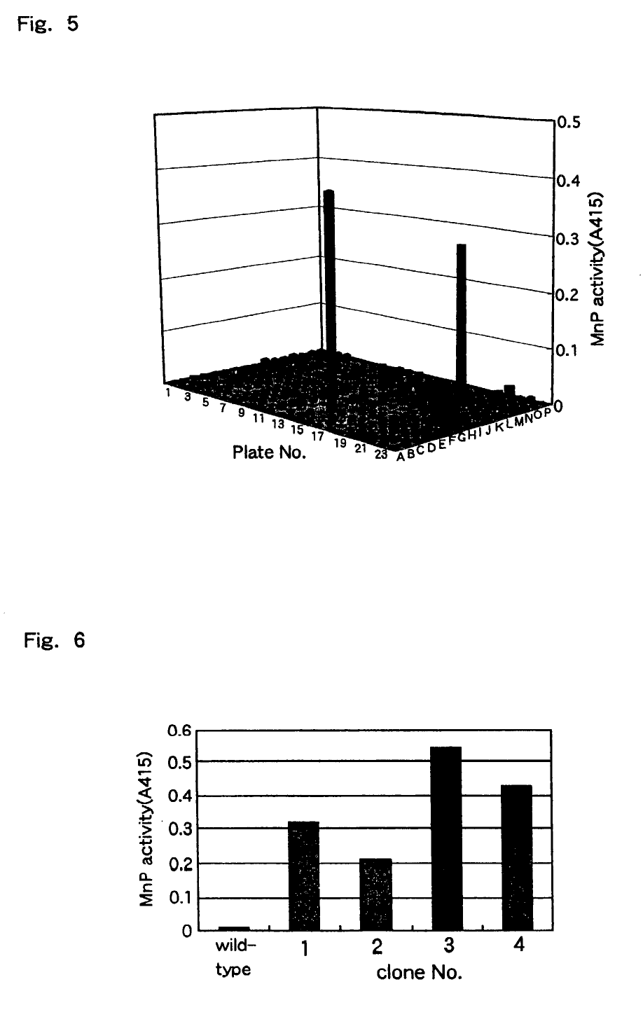

The results of the screening by HTS of the synthesis products in the present Example in a 384-well plate (1-24 rows×A-P rows) are shown in FIG. 5. As shown in the figure, positive clones could be detected in a reliable manner.

Furthermore, screening was done in 16 plates, each plate with 384 wells (6144 wells). Consequently, four primary positive clones with the MnP activity were obtained after incubation at 60° C. for 25 minutes. FIG. 6 shows the MnP activity of these clones No. 1 to No. 4. As shown in the figure, MnP prior to mutation induction (wild type MnP) never exerted such activity after such temperature processing.

### EXAMPLE 5

### Screening of Mutated MnP Stabilized with Hydrogen Peroxide

A mutation library was prepared by individually converting three amino acids positioned at the inlet of the hydrogen peroxide binding pocket in MnP (alanine at position 79, asparagine at position 81, and isoleucine at position 83) to 20 types of amino acids (NNS mutation: N=A, G, C or T; S=C or G). After the mutation library was diluted to one molecule/well on average, PCR was done under the same conditions as in Example 4, using the LA Taq polymerase.

Subsequently, 3 μl each of the resulting PCR products were added as the template to 37 μl of the same cell-free protein synthesis reaction solution as used in Example 4, for transcription/translation conjugation reaction. Then, 1.5 μl each of the synthesis products were added to 100 μl of the same solution for enzyme activity assay as in Example 1-1, followed by addition of hydrogen peroxide to achieve a final concentration of 1 mM, and the MnP activity was measured by the measurement of the absorbance at 415 nm.

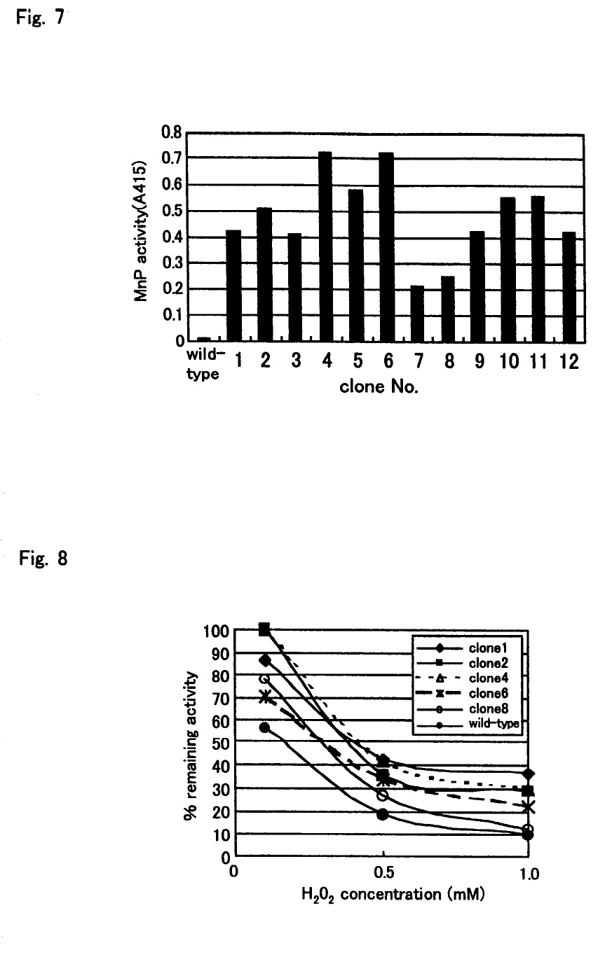

Concerning the synthesis products of the present Example, HTS was carried out, using 10 plates, each plate with 384 wells (3840 wells) in the same manner as in Example 4. FIG. 7 shows the results of the screening. As shown in the figure, 12 positive clones (clones No. 1 to No. 12 in the figure) exerting the MnP activity in the presence of hydrogen peroxide at the final concentration of 1 mM were obtained. As simultaneously shown in the figure, wild type MnP never exhibits activity after such hydrogen peroxide process.

The stability against hydrogen peroxide was subsequently assessed in detail. Specifically, the synthesis products of the transcription/translation conjugation reaction using the 12 primary positive clones were incubated in the presence of 0.1 mM, 0.5 mM and 1.0 mM hydrogen peroxide at 37° C. for 5 minutes. Subsequently, the resulting incubation mixtures produced at hydrogen peroxide concentrations of 0.5 mM and 1.0 mM were diluted with 50 mM succinic acid buffer to achieve a final hydrogen peroxide of 0.1 mM.

Then, 100 μl of the solution for enzyme activity assay as described in Example 1-1 which was without any hydrogen peroxide was added to these synthesis products with hydrogen peroxide concentrations of 0.1 mM, followed by addition of hydrogen peroxide to achieve a final concentration of 1 mM, to measure the MnP activity by measurement of the absorbance at 415 nm. Consequently, five clones Nos. 1, 2, 4, 6 and 8 among the clones No.1 through No. 12 were more stable against hydrogen peroxide, compared with wild type MnP. The residual activity of these five secondary positive clones is shown in FIG. 8, where the MnP activity after incubation in the presence of 0.1 mM hydrogen peroxide at 4° C. for 5 minutes was defined to be 100%. The amino acid sequences of these five secondary positive clones were determined and compared with the amino acid sequence of wild type MnP. The results are shown in Table 1.

TABLE 1

Amino acid No.

| Clones | | 79 | | 81 | | 83 |
|---|---|---|---|---|---|---|
| Wild type | S | A | N | N | G | I |
| Clone 1 | | E | | S | | L |
| Clone 2 | | E | | S | | L |
| Clone 4 | | S | | L | | L |
| Clone 6 | | S | | S | | L |
| Clone 8 | | E | | L | | L |

In Table 1, the wild type MnP is expressed as “without mutation”. A part of the amino acid sequence thereof is described in the column “amino acid sequence” according to the one letter expression. In the partial amino acid sequences, the “A” in the second row from left at position 79 is alanine; the “N” in the fourth row from left at position 81 is asparagine; and the “I” in the sixth row from left at position 83 is isoleucine. As indicated in the table, it is shown that in all the secondary positive clones, (a) the alanine at position 79 is converted to glutamic acid or serine; (b) the asparagine at position 81 is converted to serine or leucine; and (c) the isoleucine at position 83 is converted to leucine.

### Assessment of the Examples

As is shown in the individual Examples, in accordance with the invention, metalloproteins or proteins with intermolecular and/or intramolecular disulfide bonds, which are active form, can effectively be produced using cell-free extract solutions. Furthermore, the resulting proteins are applicable to the bioinformatics of the present invention.

While the preferred embodiments have been described, variations thereto will occur to those skilled in the art within the scope of the present inventive concepts, which are delineated by the following claims.

What is claimed is:

1. A method for protein synthesis comprising: adding a template gene of a protein with intermolecular and/or intramolecular disulfide bonds to a cell-free extract solution; and synthetically preparing the protein with intermolecular and/or intramolecular disulfide bonds using the cell-free extract solution.

2. A method for protein synthesis according to claim 1, further comprising adding at least one protein with a chaperone function together with the template gene to the cell-free extract solution.

3. A method for protein synthesis according to claim 2, wherein the protein with the chaperone function is PDI.

4. A method for protein synthesis according to claim 2, wherein the protein with the chaperone function is DnaK, DnaJ, GroEL, GroES, GrepE or Tx.

5. A method for protein synthesis according to claim 1, wherein reducing agents are eliminated from the cell-free extract solution.

6. A method for protein synthesis according to claim 2, wherein reducing agents are eliminated from the cell-free extract solution.

7. A method for protein synthesis according to claim 1, wherein the protein with intermolecular and/or intramolecular disulfide bonds is an antibody.

8. A method for protein synthesis according to claim 7, wherein the antibody is obtained in the cell-free extract solution in an active form at a concentration such that the activity of the antibody can be assayed as it is in the cell-free extract solution.

9. A method for protein synthesis according to claim 1, wherein the protein with intermolecular and/or intramolecular disulfide bonds is a metalloprotein.

10. A method for protein synthesis according to claim 9, further comprising adding a functional unit containing a metal together with the template gene to the cell-free extract solution.

11. A method for protein synthesis according to claim 10, wherein at least a part of the metalloprotein is obtained in an active form exhibiting a specific function in which the functional unit containing a metal is involved.

12. A method for protein synthesis according to claim 10, wherein the functional unit containing a metal is a metal complex.

13. A method for protein synthesis according to claim 12, wherein the metal complex is hemin.

14. A method for protein synthesis according to claim 9, wherein the metalloprotein is a heme-containing enzyme.

15. A method for protein synthesis according to claim 14, wherein the heme-containing enzyme is peroxidase.

16. A method for protein synthesis according to claim 9, wherein the metalloprotein is obtained in the cell-free extract solution in an active form at a concentration such that the activity of the metalloprotein can be assayed as it is in the cell-free extract solution.

17. A method for protein synthesis according to claim 1, wherein the cell-free extract solution is derived from Escherichia coli.

18. A method for protein screening comprising: adding to a cell-free extract solution a template gene prepared by subjecting the nucleotide sequence of nucleic acid encoding an arbitrary protein with intermolecular and/or intramolecular disulfide bonds to a specific or random modification; synthetically preparing the protein encoded by the template gene using the cell-free extract solution; and screening the synthetically prepared protein by assaying the activity thereof.

19. A retrieval method for protein function comprising: adding to a cell-free extract solution a template gene encoding an arbitrary protein requiring the formation of a specific pattern of intermolecular and/or intramolecular disulfide bonds for the expression of a function, wherein at least a part of the function is unknown; synthetically preparing the protein encoded by the template gene using the cell-free extract solution; and retrieving the unknown function of the protein by testing the function of the prepared protein.

20. A retrieval method for protein function according to claim 19, wherein the protein is a protein composing a subunit of a protein complex with an arbitrary function.

21. A retrieval method for protein function according to claim 20, wherein the protein complex is an antibody.
